(19) United States
(12) Patent Application Publication
(10) Pub. No.: US 2014/0350534 A1
(43) Pub. Date: Nov. 27, 2014

(54) RAMAN BASED ABLATION/RESECTION SYSTEMS AND METHODS

(71) Applicant: Sloan-Kettering Institute For Cancer Research, New York, NY (US)

(72) Inventors: Moritz Kircher, New York, NY (US); Ricardo Toledo-Crow, New York, NY (US)

(73) Assignee: Sloan-Kettering Institute For Cancer Research, New York, NY (US)

(21) Appl. No.: 14/184,934

(22) Filed: Feb. 20, 2014

Related U.S. Application Data
(60) Provisional application No. 61/767,241, filed on Feb. 20, 2013.

(51) Int. Cl. A61B 18/20; A61B 5/00; A61B 18/08

(52) U.S. Cl. CPC A61B 18/203; A61B 18/082; A61B 5/0075; A61B 5/0071; A61B 2018/00773
USPC 606/10; 606/41

(57) ABSTRACT

Described herein are methods, systems, and devices for automated laser ablation and/or tissue resection triggered by Raman spectroscopic information. These systems and methods provide for precise removal of cancerous or other diseased tissue with minimal damage to adjacent healthy tissue.

### CROSS REFERENCE TO RELATED APPLICATIONS

This application claims the benefit of U.S. Provisional Application No. 61/767,241 filed Feb. 20, 2013, the contents of which are hereby incorporated by reference herein in their entirety.

### BACKGROUND

A variety of surgical techniques have been developed for the physical removal of cancerous or other diseased tissue. A goal of these methods is to remove cancerous/diseased tissue with minimal damage to nearby healthy tissue. A surgeon resects tissue that appears to be abnormal from visual inspection.

Although advances in medical imaging have been made to help a surgeon localize abnormal tissue prior to surgery, the surgeon's ability to identify abnormal tissue at the margins of infiltratively growing cancers or in the setting of metastatis spread via visual inspection are limited. There remains a need for systems and methods that precisely remove cancerous and/or diseased tissue from locations within, surrounding, and/or adjacent to critical organs or tissue, where significant harm may result from damage to or removal of healthy tissue.

### SUMMARY

Systems and methods are presented herein that provide automated laser ablation and/or tissue resection triggered by detection of one or more Raman reporters, such as Raman nanoparticles (e.g., surface-enhanced Raman spectroscopic (SERS) and/or surface-enhanced (resonance) Raman spectroscopic (SERRS) nanoparticles), and/or intrinsic species that produce(s) a characteristic, identifiable Raman signal (e.g., Raman spectrum). These systems and methods provide for precise removal of cancerous or other diseased tissue with minimal damage to adjacent healthy tissue.

A system is provided herein with a resection/ablation mechanism that is activated only at locations at which one or more Raman reporters are detected. For example, an ablation laser or resection mechanism is activated at a location only when a Raman signal indicative of the presence of a Raman reporter at the location is recognized by a Raman spectrometer, where the Raman reporter is associated with tissue to be resected/ablated (e.g., cancerous, diseased, infected, or otherwise abnormal tissue). If the specific Raman signal associated with one or more Raman reporters is not detected, the ablation/resection mechanism is not activated. In this way, extremely precise destruction and/or removal of diseased tissue may be accomplished while limiting damage to nearby healthy tissue. For example, a precision of 500, 400, 300, 200, 100, or 50 micrometers or better may be achieved.

In certain embodiments, a Raman reporter is a Raman nanoparticle (e.g., SERS and/or SERRS nanoparticle), or a component of a Raman nanoparticle. In some embodiments, Raman nanoparticles are administered (e.g., by injection or topically) to a patient/subject and are allowed to accumulate in and/or around cancerous tissue, pre-cancerous tissue, or other diseased tissue (e.g., necrotic tissue, infected tissue, inflamed tissue, etc.). The Raman nanoparticles that may be used in the disclosed systems and methods include, for example, those described in Kircher et al., Nature Medicine 2012 Apr. 15; 18(5): 829-34, the text of which is incorporated herein by reference in its entirety. These are based on surface enhanced Raman scattering (SERS). Other nanoparticles may be used, as long as they create a sufficiently detectable and distinguishable Raman signal (e.g., a Raman spectrum).

In some embodiments, a Raman reporter is a molecule or substance present within, on, or near diseased tissue itself (“intrinsic species”), which is identified or targeted using an intrinsic Raman spectrum (e.g., a Raman spectrum detected following illumination of tissue). In some embodiments, tissue is selected and/or resected/ablated if a detected Raman signal satisfactorily matches a predetermined Raman signal known to be indicative of the Raman reporter.

In certain embodiments, the system includes a hand-held instrument of size and shape that may be customized depending on the application. For example, the system may include a laser suitable to ablate/destroy tissue (such as, for example, a CO₂ or Nd:YAG laser). Alternatively or additionally, the system may include a motor-driven, controlled resection mechanism such as, for example, a small rotating blade, located at the tip of the hand-held instrument. Alternatively or additionally, the system may include an electro-cautery mechanism, a cryoablation mechanism, and/or a radiofrequency ablation mechanism. In some embodiments, an ablation mechanism is a robotic/remote controlled ablation mechanism (e.g., located at the tip of the hand-held instrument). The system may also include a vacuum suction mechanism connected to a collection bag for removal of destroyed/ablated/resected tissue as well as nanoparticles located within the target tissue. The system may also include an excitation laser and associated optics for determination of Raman spectra associated with detected photons emanating from the tissue. A rinsing mechanism may be included to keep optics clean during the procedure. The hand-held instrument may be connected to other components of the system via fiberoptic cable, for example, and suction tubing. The hand-held instrument may be connected to components of a box housing mechanics, optics, electronics, excitation laser, ablation laser, resection instrument motor, radiofrequency or cryoablation generator, suction motor, rinsing mechanism, Raman spectral analysis optics, and/or the CCD chip.

A surgeon using the disclosed system can destroy or remove cancerous (or otherwise abnormal) tissue quickly and with high precision in a semiautomated fashion. For example, the hand-held instrument may be positioned and moved over regions of tissue “blindly” or “semi-blindly” near the site of disease/cancer, as the system destroys only cancerous tissue, with no or minimal damage to adjacent healthy tissue. The system may be used, for example, during open surgical procedures, in-office (non-surgical) procedures, invasive procedures, non-invasive or minimally invasive procedures, endoscopic procedures, robotically-assisted procedures, or in external applications such as skin cancer removal.

An automated or semi-automated X-Y (two-dimensional) or X-Y-Z (three-dimensional) scan of the tissue by the instrument may be performed. For example, the detection+ablation/resection instrument may be positioned such that excitation light from the instrument is directed to a sequence of X-Y or X-Y-Z positions of the tissue. At each location, light is detected and the processor of the system determines whether a Raman reporter is detected at that location. If so, the resection/ablation mechanism is activated at that location such that only tissue at that location is removed or destroyed. The resection/ablation mechanism is then deactivated prior to moving the instrument to a second location, whereupon excitation light is directed to the second position and light is detected from the second position and the resection/ablation mechanism is activated only if a Raman reporter is detected at that second position, and so on.

For applications involving skin cancer removal, or other abnormal topical tissue removal, a Raman reporter is a SERS nanoparticle (or a component thereof) that may be applied topically or injected prior to operation of the hand-held instrument. A topical application may include penetrating peptides to facilitate absorption of the SERS nanoparticles into the skin. In some embodiments, a Raman reporter is an intrinsic species within, on, or near the skin cancer or other abnormal tissue.

In one aspect, the invention encompasses a system comprising: an excitation light source for directing excitation light onto or into a target tissue; an instrument (e.g., hand-held instrument) operably linked to the excitation light source, the instrument comprising: optics for directing the excitation light onto or into the target tissue; a detector for detecting Raman scattered photons emanating from the target tissue, said Raman scattered photons resulting from illumination with the excitation light; a resector/ablator mechanism; a processor (e.g., a Raman spectrometer and associated computer processor and/or software) configured to process data corresponding to the Raman scattered photons detected from the target tissue; and a resector/ablator controller operably linked to the processor and operably linked to the resector/ablator mechanism.

In certain embodiments, the excitation light source is a laser. In certain embodiments, the excitation light has a wavelength of about 500 nm to about 10 μm. In some embodiments, the excitation light has a wavelength of about 785 nm. In certain embodiments, the excitation light is near-infrared light (e.g., where deeper penetration, e.g., up to about 1 cm, is desired). In certain embodiments, the excitation is ultraviolet light (e.g., where shallow penetration, e.g., only up to 1 mm, up to 2 mm, or up to 3 mm, is desired). In certain embodiments, the instrument is an endoscopic instrument.

In certain embodiments, the resector/ablator mechanism comprises a laser. In certain embodiments, the laser of the resector/ablator mechanism is a CO₂ laser. In certain embodiments, the resector/ablator mechanism is a mechanical resector (e.g., rotary blade, vibrating knife, or percussing knife). In some embodiments, the resector/ablator mechanism is an electro-cautery mechanism, a cryoablation mechanism, and/or a radiofrequency ablation mechanism. In certain embodiments, the resector/ablator controller is configured to activate the resector/ablator mechanism to resect, ablate, and/or destroy tissue at a given location only if Raman scattered photons detected from the given location (e.g., a detected Raman signal or spectrum) indicate the presence of a Raman reporter (e.g., SERS nanoparticles, SERRS nanoparticles, or an intrinsic species). In certain embodiments, the system further comprises a suction vacuum operably linked to the instrument.

In another aspect, the invention encompasses a method of resecting, ablating, and/or destroying diseased tissue, the method comprising the steps of: positioning an instrument in relation to a first location (e.g., (x,y,z) or (x,y) location) of a target tissue of a subject (e.g., human or animal), the instrument comprising: optics for directing excitation light onto or into the target tissue at a given location; a detector for detecting Raman scattered photons emanating from the target tissue at the given location; and a resector/ablator mechanism; detecting the Raman scattered photons emanating from the first location of the target tissue; analyzing the detected Raman scattered photons emanating from the first location to determine whether the detected photons are indicative of the presence of a Raman reporter (e.g., SERS nanoparticles, SERRS nanoparticles, or intrinsic species) at the first location; and activating the resector/ablator mechanism (e.g., via a resector/ablator controller) to resect the target tissue at the first location only if the analyzed photons from the first location are determined to be indicative of the presence of a Raman reporter at the first location.

In certain embodiments, the method further comprises: deactivating the resector/ablator mechanism prior to repositioning of the instrument in relation to a second location of the target tissue (e.g., wherein the second location of the target tissue is adjacent to the first location); detecting the Raman scattered photons emanating from the second location of the target tissue; analyzing the detected Raman scattered photons emanating from the second location to determine whether the detected photons are indicative of the presence of a Raman reporter (e.g., SERS nanoparticles, SERRS nanoparticles, and/or intrinsic species) at the second location; and activating the resector/ablator mechanism to resect, ablate, and/or destroy the target tissue at the second location only if the analyzed photons from the second location are determined to be indicative of the presence of the Raman reporter at the second location.

In certain embodiments, the method further comprises administering nanoparticles (e.g., SERS nanoparticles or SERRS nanoparticles) to the subject prior to implementation of the instrument (e.g., allowing accumulation of the nanoparticles in regions associated with disease). In certain embodiments, the method further comprises scanning the subject prior to implementation of the instrument to confirm the absence of nanoparticles from healthy (e.g., normal, e.g., non-cancerous) tissue.

In certain embodiments, the instrument is operably linked to an excitation light source. In certain embodiments, the excitation light source is a laser. In certain embodiments, the excitation light has a wavelength of about of about 500 nm to about 10 μm. In some embodiments, the excitation light has a wavelength of about 785 nm. In certain embodiments, the excitation light is near-infrared light (e.g., where deeper penetration, e.g., up to about 1 cm, is desired). In certain embodiments, the excitation is ultraviolet light (e.g., where shallow penetration, e.g., only up to 1 mm, up to 2 mm, or up to 3 mm, is desired). In certain embodiments, the instrument is an endoscopic device. In certain embodiments, the resector/ablator mechanism comprises a laser. In certain embodiments, the laser of the resector/ablator mechanism is a CO₂ laser. In certain embodiments, the resector/ablator mechanism is a mechanical resector (e.g., rotary blade, vibrating knife, or percussing knife). In some embodiments, the resector/ablator mechanism is an electro-cautery mechanism, a cryoablation mechanism, and/or a radiofrequency ablation mechanism.

In certain embodiments, the analyzing step comprises using a computer processor (e.g., a Raman spectrometer and associated computer processor and/or software) to process data corresponding to the detected Raman scattered photons. In certain embodiments, the method further comprises removing resected tissue. In certain embodiments, the method is an in vivo method.

In any of the aspects described herein, the instrument can be a handheld instrument, a stationary instrument, and/or a robotically assisted instrument. In some embodiments, the device is an endoscopic instrument.

In any of the aspects described herein, the system may further include other optics, hardware, electronics, and/or software for imaging target cells or tissues.

### BRIEF DESCRIPTION OF THE DRAWINGS

The following figures are presented for the purpose of illustration only, and are not intended to be limiting.

FIG. 1 is a schematic illustration of steps of an exemplary method of the disclosure.

FIG. 2 is a schematic illustration of an exemplary system of the disclosure.

FIG. 3 is a schematic illustration of an exemplary system of the disclosure.

FIG. 4 is a schematic illustration of a system for controlling a Raman scanner according to the disclosure.

All publications, patent applications, patents, and other references mentioned herein, are incorporated by reference in their entirety. In case of conflict, the present specification, including definitions, will control. In addition, the materials, methods, and examples are illustrative only and not intended to be limiting. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below.

Other features and advantages of the invention will be apparent from the following detailed description, and from the claims.

### DETAILED DESCRIPTION

The present disclosure encompasses methods, systems, and devices for assessing and/or treating (e.g., ablating and/or resecting) cells and/or tissue in a subject. In particular, the methods and devices described herein provide for detection of Raman spectra from cells and/or tissues and subsequent targeted ablation and/or resection of cells and/or tissues from which Raman spectra are detected. In some embodiments, methods, systems and devices of the disclosure do not need or include components to image target cells and/or tissues. In some embodiments, systems and devices of the disclosure further include components to image target cells and/or tissues.

In some embodiments, the disclosure encompasses an automated surgical tissue resection instrument and/or an automated laser ablation instrument that resects and/or ablates only disease tissue at locations at which a Raman reporter is detected, e.g., by comparing detected Raman signal to specific Raman signals/spectra associated with one or more type of Raman nanoparticle or intrinsic species known to be associated with the presence of tissue to be resected or ablated. Such an instrument resects and/or ablates only diseased tissue, because a motorized resection mechanism and/or ablation laser included in the instrument is activated only when the specific spectrum of a Raman reporter is recognized by a Raman spectrometer included in the system. If a specific Raman signal is not detected at a given location (indicating healthy tissue), the instrument automatically stops (or does not start) resecting and/or ablating at that location. In some embodiments, a Raman reporter is a Raman nanoparticle, which can optionally can be designed to target and/or accumulate within or proximate to diseased tissue of interest (e.g., cancer, infection, or inflammation).

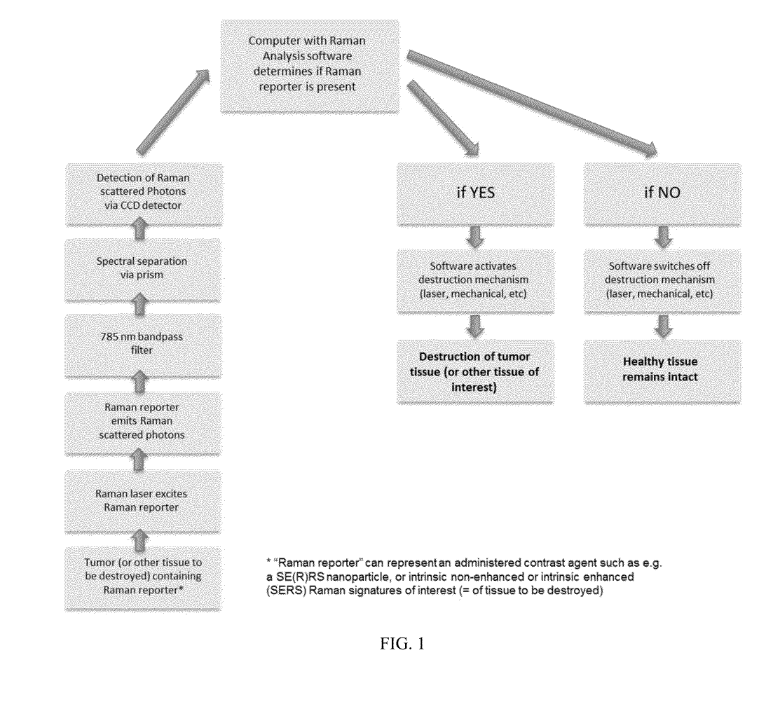

FIG. 1 depicts a flowchart of an exemplary method of the disclosure. Starting at the lower left box, a diseased tissue (e.g., a tumor) containing a Raman reporter (e.g., a Raman nanoparticle described herein or an intrinsic Raman species) is provided. In some embodiments, a Raman nanoparticle is administered to a subject, and the nanoparticle accumulates within diseased tissue. Using a Raman laser, a Raman reporter present within the diseased tissue is excited, which emits Raman scattered photons. In this exemplary method, Raman scattered photons are filtered using a 785 nm bandpass filter and are spectrally separated using a prism. Raman scattered photons are detected using a detector, e.g., a CCD detector. Detected Raman scattered photons are then analyzed using an analyzer (e.g., a computer with Raman analysis software) to determine if a Raman reporter is present. If a Raman reporter is present, the analyzer activates a resector/ablation mechanism (e.g., a mechanical resector (e.g., rotary blade, vibrating knife, or percussing knife), an electro-cautery mechanism, a cryoablation mechanism, and/or a radiofrequency ablation mechanism), which destroys diseased tissue. If the analyzer determines that no Raman reporter is present, the analyzer does not activate (or, if previously activated, shuts off) the resector mechanism, preserving healthy tissue. In some embodiments, a Raman reporter is initially detected, and the steps of excitation, detection, and analysis are repeated until a Raman reporter is not detected.

In some embodiments, systems and devices of the disclosure enable more precise resection and/or ablation of diseased tissue. Surgeons often resect diseased tissue by visual inspection, which may be imprecise at the margins of diseased and non-diseased tissue, for example, at margins of infiltratively growing cancers or in the setting of metastic spread. In some embodiments, a Raman reporter is a Raman nanoparticle, which specifically targets diseased tissue (e.g., cancer), methods, systems, and devices of the disclosure can allow a surgeon to resect and/or ablate diseased tissue (e.g., cancer) faster and with much higher precision, e.g., compared to visual inspection or other known methods. In some embodiments, a Raman reporter is an intrinsic species within, on, or near diseased tissue, and a predetermined intrinsic Raman spectrum is used in the methods described herein. In some embodiments, resection and/or ablation is performed in a semiautomated fashion, e.g., a device described herein is held approximately at or moved generally over a site of disease and automatically removes only diseased tissue but not adjacent healthy tissue. The methods, systems, and devices described herein have many applications, e.g., open surgical applications, endoscopic approaches, and robotically assisted approaches.

### Raman Spectroscopy

Raman spectroscopy provides information about the vibrational state of molecules. Many molecules have atomic bonds capable of existing in a number of vibrational states. Such a molecule is able to absorb incident radiation that matches a transition between two of its allowed vibrational states and to subsequently emit the radiation. These vibrational transitions exhibit characteristic energies that permit definition and characterization of the bonds that are present in a compound. Analysis of vibrational transitions therefore permits spectroscopic molecular identification.

Most often, absorbed radiation is re-radiated at the same wavelength, a process designated Rayleigh or elastic scattering. In some instances, the re-radiated radiation can contain slightly more or slightly less energy than the absorbed radiation (depending on the allowable vibrational states and the initial and final vibrational states of the molecule). The energy difference is consumed by a transition between allowable vibrational states, and these vibrational transitions exhibit characteristic values for particular chemical bonds, which accounts for the specificity of vibrational spectroscopies such as Raman spectroscopy.

The result of the energy difference between the incident and re-radiated radiation is manifested as a shift in the wavelength between the incident and re-radiated radiation, and the degree of difference is designated the Raman shift (RS), measured in units of wavenumber (inverse length). If the incident light is substantially monochromatic (single wavelength) as it is when using a laser source, the scattered light that differs in frequency can be more easily distinguished from Rayleigh scattered light.

Raman spectroscopy may utilize high efficiency solid-state lasers, efficient laser rejection filters, and silicon CCD detectors. In general, the wavelength and bandwidth of light used to illuminate a sample is not critical, so long as the other optical elements of the system operate in the same spectral range as the light source.

In general, a sample should be irradiated with monochromatic light (e.g., substantially monochromatic light). Suitable light sources include various lasers and polychromatic light source-monochromator combinations. It is recognized that the bandwidth of the irradiating light, resolution of the wavelength resolving element(s), and the spectral range of a detector determine how well a spectral feature can be observed, detected, or distinguished from other spectral features. The combined properties of these elements (e.g., the light source, the filter, grating, or other mechanism used to distinguish Raman scattered light by wavelength) define the spectral resolution of the Raman signal detection system. The known relationships of these elements enable the skilled artisan to select appropriate components in readily calculable ways. Limitations in spectral resolution of the system (e.g., limitations relating to the bandwidth of irradiating light, grating groove density, slit width, interferometer stepping, and other factors) can limit the ability to resolve, detect, or distinguish spectral features. The separation and shape of Raman scattering signals can be used to determine the acceptable limits of spectral resolution for the system for any Raman spectral features.

Typically, a Raman peak that both is distinctive of a substance of interest (e.g., a Raman nanoparticle or intrinsic species described herein) and exhibits an acceptable signal-to-noise ratio can be selected. Multiple Raman shift values characteristic of the substance (e.g., Raman nanoparticle or intrinsic species) can be assessed, as can the shape of a Raman spectral region that may include multiple Raman peaks.

### Raman Nanoparticles

In some embodiments, methods of the disclosure include use of Raman nanoparticles, e.g., surface-enhanced Raman scattering (SERS) nanoparticles or surface-enhanced (resonance) Raman scattering (SERRS) nanoparticles. SERS and SERRS refer to an increase in Raman scattering exhibited by certain molecules in proximity to certain metal surfaces (see, U.S. Pat. No. 5,567,628; McNay et al., Applied Spectroscopy 65:825-837 (2011)). The SERS effect can be enhanced through combination with a resonance Raman effect. The SERS effect can be increased by selecting a frequency for an excitation light that is in resonance with a major absorption band of a molecule being illuminated. In short, a significant increase in the intensity of Raman light scattering can be observed when molecules are brought into close proximity to (but not necessarily in contact with) certain metal surfaces. Metal surfaces can be roughened or coated with minute metal particles. The increase in intensity can be on the order of several million-fold or more.

Nanoparticles that can be detected using Raman spectroscopy can be used in the methods and devices described herein. Raman nanoparticles and SERS nanoparticles and methods of their production are known and described in, e.g., U.S. Publ. No. 2012/0179029; Kircher et al., Nature Med. 18:829-834 (2012); Yigit et al., Am. J. Nucl. Med. Mol. Imaging 2:232-241 (2012); Zhang et al., Small. 7:3261-9 (2011); Zhang et al., Curr. Pharm. Biotechnol. 11:654-661 (2010).

In some embodiments, Raman nanoparticles (e.g., SERS nanoparticles) are administered to a subject having or suspected of having cancer. Without being bound to theory, it is believed that such nanoparticles target to and/or accumulate within, on the surface of, or proximate to cancer cells by enhanced permeability and retention (EPR) as described in, e.g., Kircher et al., Nature Med. 18:829-834 (2012); and Adiseshaiah et al., Wiley Interdiscip. Rev. Nanomed. Nanobiotechnol. 2:99-112 (2010). Thus, detection of Raman nanoparticles indicates such cells and/or tissues are cancerous.

In some embodiments, Raman reporter detection is combined with one or more additional modalities for identification of tissue to be resected or ablated. For example, Raman reporter detection can be combined with video imaging, MRI, NMR, PET, SPECT, CT, X-ray, ultrasound, photoacoustic detection, and/or fluorescent detection, for example. Also, Raman nanoparticles may be designed such that they are detected by reporter detection combined with one or more other modalities, such as video imaging, MRI, NMR, PET, SPECT, CT, X-ray, ultrasound, photoacoustic detection, and/or fluorescent detection, for example. Such nanoparticles are described in, e.g., Kircher et al., Nature Med. 18:829-834 (2012).

### Targeting Agents

In some embodiments, Raman nanoparticles described herein include one or more targeting agent to facilitate and/or enhance the targeting of nanoparticles to a diseased tissue. Targeting agents include, e.g., various specific ligands, such as antibodies, monoclonal antibodies and their fragments, folate, mannose, galactose and other mono-, di-, and oligosaccharides, and RGD peptide. Additional examples of targeting agents include, but are not limited to, nucleic acids (e.g., RNA and DNA), polypeptides (e.g., receptor ligands, signal peptides, avidin, Protein A, and antigen binding proteins), polysaccharides, biotin, hydrophobic groups, hydrophilic groups, drugs, and any organic molecules that bind to receptors.

In some embodiments, a targeting agent is an antigen binding protein (e.g., an antibody or binding portion thereof). Antibodies can be generated using known methods to allow for the specific targeting of antigens or immunogens (e.g., tumor, tissue, or pathogen specific antigens) on various biological targets (e.g., pathogens, or tumor cells). Such antibodies include, but are not limited to, polyclonal antibodies; monoclonal antibodies or antigen binding fragments thereof; modified antibodies such as chimeric antibodies, reshaped antibodies, humanized antibodies, or fragments thereof (e.g., Fv, Fab′, Fab, F(ab′)₂); or biosynthetic antibodies, e.g., single chain antibodies, single domain antibodies (DAB), Fvs, or single chain Fvs (scFv). Methods of making and using polyclonal and monoclonal antibodies are well known in the art, e.g., in Harlow et al., Using Antibodies: A Laboratory Manual: Portable Protocol I. Cold Spring Harbor Laboratory (Dec. 1, 1998). Methods for making modified antibodies and antibody fragments (e.g., chimeric antibodies, reshaped antibodies, humanized antibodies, or fragments thereof, e.g., Fab′, Fab, F(ab′)₂ fragments); or biosynthetic antibodies (e.g., single chain antibodies, single domain antibodies (DABs), Fv, single chain Fv (scFv), and the like), are known in the art and can be found, e.g., in Zola, Monoclonal Antibodies: Preparation and Use of Monoclonal Antibodies and Engineered Antibody Derivatives, Springer Verlag (Dec. 15, 2000; 1st edition).

In some embodiments, the targeting agent is a nucleic acid (e.g., RNA or DNA). In some examples, the nucleic acid targeting agents are designed to hybridize by base pairing to a particular nucleic acid (e.g., chromosomal DNA, mRNA, or ribosomal RNA). In other situations, the nucleic acids bind a ligand or biological target. For example, the nucleic acid can bind reverse transcriptase, Rev or Tat proteins of HIV (Tuerk et al., Gene 137:33-9 (1993)); human nerve growth factor (Binkley et al., Nuc. Acids Res. 23:3198-205 (1995)); or vascular endothelial growth factor (Jellinek et al., Biochem. 83:10450-10456 (1994)). Nucleic acids that bind ligands can be identified by known methods, such as the SELEX procedure (see, e.g., U.S. Pat. Nos. 5,475,096; 5,270,163; and 5,475,096; and WO 97/38134; WO 98/33941; and WO 99/07724). The targeting agents can also be aptamers that bind to particular sequences.

The targeting agents can recognize a variety of known epitopes on preselected biological targets (e.g., pathogens or tumor cells). In some embodiments, the targeting agent targets nanoparticles to factors expressed by oncogenes. These can include, but are not limited to, tyrosine kinases (membrane-associated and cytoplasmic forms), such as members of the Src family; serine/threonine kinases, such as Mos; growth factor and receptors, such as platelet derived growth factor (PDDG), small GTPases (G proteins), including the ras family, cyclin-dependent protein kinases (cdk), members of the myc family members, including c-myc, N-myc, and L-myc, and bcl-2 family members.

### Systems and Instruments

Systems of the disclosure include detectors and associated components for detecting Raman spectra from cells and/or tissues and implements for treating (e.g., ablating and/or resecting) cells and/or tissues from which Raman spectra are detected. In some embodiments, such systems include an excitation source (e.g., a light source), optics for directing such excitation source to a sample (e.g., cells and/or tissues), a detector for detecting Raman spectra from such sample, and implements for treating (e.g., ablating and/or resecting) cells and/or tissues from which Raman spectra are detected.

In some embodiments, a system of the disclosure includes a handheld instrument of size and length that can be customized to a particular application. A system can include a resector/ablation mechanism (e.g., a mechanical resector (e.g., rotary blade, vibrating knife, or percussing knife), an electro-cautery mechanism, a cryoablation mechanism, and/or a radiofrequency ablation mechanism. A system can optionally include a vacuum suction mechanism connected to a collection bag that removes resected tissue from the site of resection. Adjacent and/or near the motorized resection mechanism within the handheld device can be located an excitation laser pathway and optics for measuring emitted Raman spectra. Optionally, a rinsing mechanism can be included within the device to help clean the optics. The hand-held device can be connected with a cable (e.g., fiberoptic cable) and tubing (e.g., suction tubing) to a box located adjacent to the operating site that houses mechanics, optics, and electronics (e.g., excitation laser, Raman spectral analysis optics, CCD chips, and optionally motors to drive the resection instrument, suction motor, and rinsing mechanism).

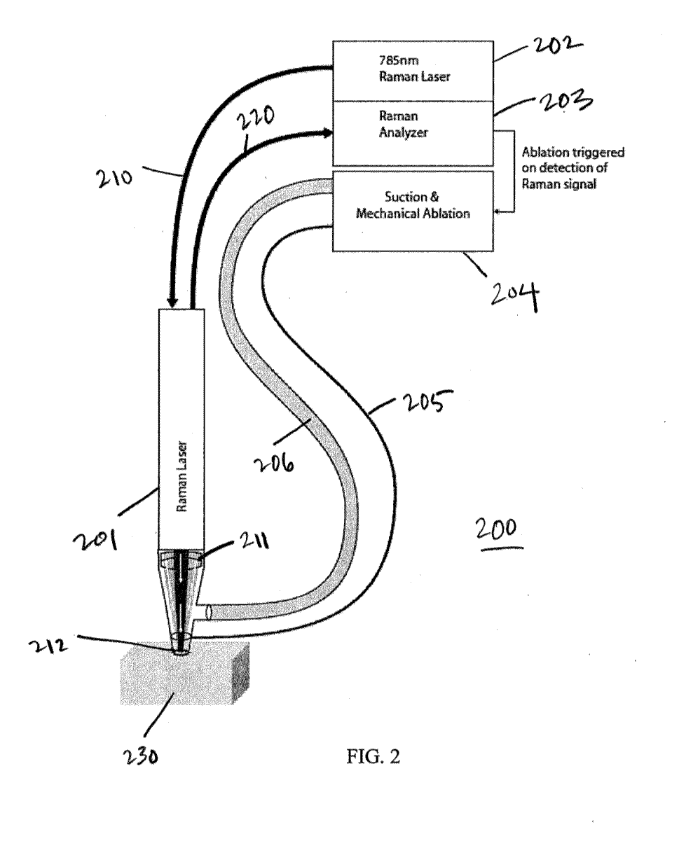

An exemplary system is illustrated schematically in FIG. 2. As shown in FIG. 2, system 200 of the disclosure includes a hand-held instrument/housing 201 having a terminal end 212. The instrument 201 may include optics for directing an excitation light onto a target sample 230 (e.g., cells, or tissue). In this exemplary system, excitation light source 202 is a Raman laser, for example, having a wavelength of 785 nm. The excitation light is transmitted along cable 210 from excitation light source 202 through device 201 and is directed to target tissue 230 through terminal end 212. In some embodiments, the excitation light passes through one or more filters 211 before reaching target 230. The filter(s) may or may not be contained within the hand-held instrument 201. In alternative embodiments, the excitation light is not directed onto the tissue 230 by the hand-held instrument 201, but instead is directed onto the tissue 230 via optics, apart from the instrument 201.

The system 200 also includes a detector for detecting a signal from target 230. Such signal follows cable 220 to signal analyzer 203. In this exemplary system, signal analyzer 203 is a Raman analyzer. Upon determination that an appropriate signal is detected, signal analyzer 203 relays a positive signal to ablation controller 204. Ablation controller 204 is operably linked to instrument 201 via cable 205, which terminates in an ablation device near terminal end 212 of instrument 201. Upon receiving a positive signal from ablation controller 204, the ablation device ablates cells and/or tissue at or near target 230. In some embodiments, ablation controller 204 includes a mechanical ablation controller operably linked to a suction vacuum mechanism near terminal end 212 of instrument 201 via tubing 206.

In alternative embodiments, the system 200 includes a motor-driven and controlled resection mechanism (e.g., a rotating blade) located at the tip 212 of the handheld device 201, such that activation of the resection mechanism is triggered upon detection of a Raman signal by the Raman Analyzer 203.

In some embodiments, a system of the disclosure includes a handheld instrument of size and length that can be customized depending on application. A system can include a laser suitable for ablating/destroying tissue (e.g., a CO₂ or Nd:YAG laser). A system can optionally include a vacuum suction mechanism connected to a collection bag that removes destroyed tissue (and, optionally, nanoparticles described herein) within targeted tissue. Adjacent to the ablation laser pathway within the handheld device can be located an excitation laser pathway and optics for measuring emitted Raman spectra. Optionally, a rinsing mechanism can be included within the device to help clean the optics. The handheld device can be connected with a cable (e.g., fiberoptic cable) and tubing (e.g., suction tubing) to a box located adjacent to the operating site that houses mechanics, optics, and electronics (e.g., excitation laser, ablation laser, Raman spectral analysis optics, CCD chip(s), and optionally motors to drive the suction motor, and rinsing mechanism).

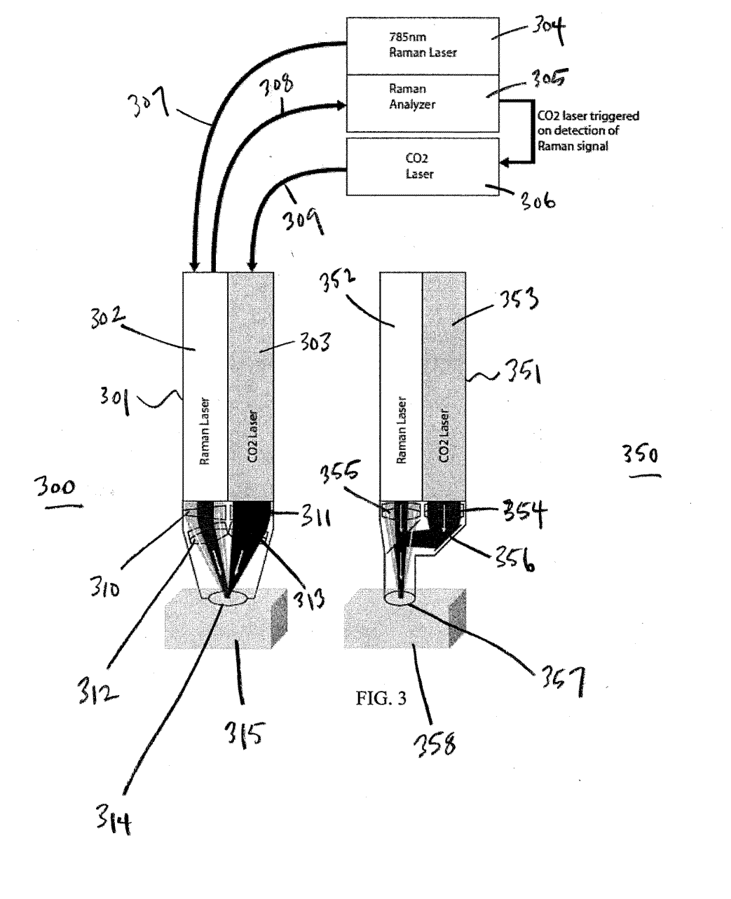

Two exemplary systems are illustrated schematically in FIG. 3. As shown in FIG. 3, system 300 of the disclosure includes a hand-held instrument 301 having a terminal end 314. The instrument 301 includes a housing 302 for directing an excitation light to a target sample 315. In this exemplary system, excitation light source 304 is a Raman laser, for example, having a wavelength of 785 nm. The excitation light is transmitted along cable 307 from excitation light source 304 through instrument 301 and is directed to target 315 through terminal end 314. In some embodiments, the excitation light passes through one or more filters 310 and 312 before reaching target 315. The filter(s) may or may not be contained within the hand-held instrument 301. In alternative embodiments, the excitation light is not directed onto the tissue 315 by the hand-held instrument 301, but instead is directed onto the tissue 315 via optics apart from the instrument 301.

The system 300 also includes a detector for detecting a signal from target 315. Such signal travels through cable 308 to signal analyzer 305. In this exemplary system, signal analyzer 305 is a Raman analyzer. Signal analyzer 305 is operably linked to ablation laser 306. In this exemplary system, ablation laser 306 is a CO₂ laser. Upon determination that an appropriate signal is detected, signal analyzer 305 relays a positive signal to ablation laser 306. Ablation laser 306 is operably linked to device 301 via cable 309, which directs the ablation laser through housing 303 to target 315. In some embodiments, ablation laser passes through filters 311 and 313 before reaching target 315.

FIG. 3 also illustrates exemplary system 350, which differs from system 300 in the configuration of device 351. As shown in FIG. 3, device 351 includes housing 352 for directing excitation light from an excitation light source and for directing Raman signals to a signal analyzer as described for system 300. Device 351 also includes housing 353 for directing ablation laser to target 358, as described for system 300. Device 351 includes filter 354 and deflector 356, which directs ablation laser along or near the same pathway used by the excitation light to reach target 358.

The instruments 201, 301, 350 described above, instead of being hand-held, may be endoscopic instruments designed for insertion into a patient, for example, into the gastrointestinal tract, the respiratory tract, the ear, the urinary tract, the female reproductive system, the abdominal or pelvic cavity, the interior of a joint (arthroscopy), organs of the chest, or the amnion.

In some embodiments, systems 200 and 300 described above additionally include one or more additional modalities for detecting a Raman nanoparticle, and/or for otherwise detecting tissue to be ablated or resected. For example, the system further includes MRI, NMR, PET, SPECT, CT, X-ray, ultrasound, photoacoustic, and/or fluorescent detection modalities.

Systems of the disclosure described herein may have components of small size (e.g., micromechanical components), such that the systems may be used in microsurgical procedures.

Systems of the disclosure described herein may be robot-assisted or robot-guided. For example, the instrument 201, 301, 350 may be part of a robotic system that positions and/or moves the instrument automatically or semi-automatically. Other components of known robotic surgical systems may be used in conjunction with the systems of this disclosure.

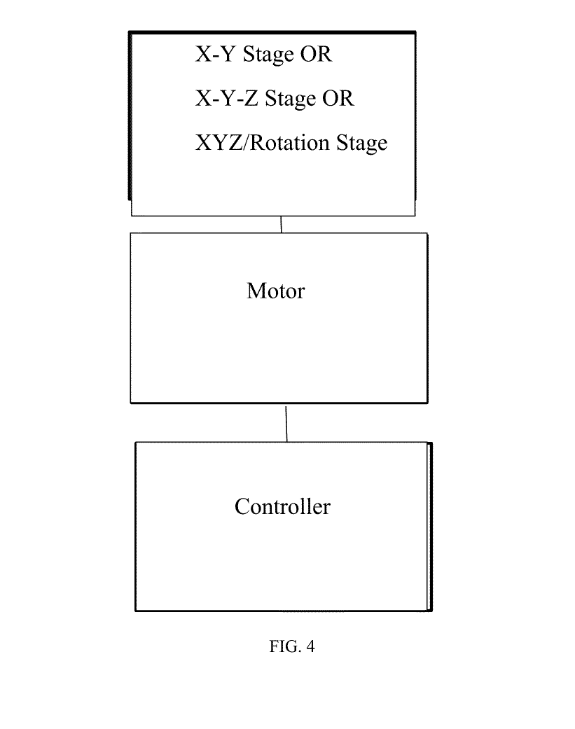

In some embodiments, a system described herein further includes a Raman raster scanning device. For example, a Raman raster scanning device can be used to scan (e.g., systematically scan) a field having a particular dimension (e.g., a surface area of target tissue). FIG. 4 illustrates an exemplary system for using a Raman scanning device, which can be used in any of the embodiments described herein. As shown in FIG. 4, a controller is operably linked to a motor, which manipulates the position of a stage (e.g., an X-Y stage, an X-Y-Z stage, or an XYZ/rotation stage).

In some embodiments, a system described herein includes a Raman scanner that allows scanning of a field of view of about 5×5 cm, 10×10 cm, 20×20 cm, or larger. In some embodiments, a Raman scanner allows scanning of a field of view of about 25 cm², 50 cm², 75 cm², 100 cm², 150 cm², 200 cm², 300 cm², 400 cm², 500 cm², or larger. In some embodiments, the Raman scanner scans a field of view in a matter of minutes, e.g., in about 1-60 minutes. A Raman scanner can include one or more lasers, e.g., one or more excitation lasers described herein, that are moved across the field of view. In some embodiments, the one or more lasers are moved across a field of view in an automated fashion.

In some embodiments, Raman spectra emitted by a Raman reporter are recorded (e.g., at an integration speed of about 10-50 ms). For example, a Raman scanner can acquire about 100, 250, 500, 750, 1000, 1250, 1500, 2000 or more spectra in about 5-60 minutes. In some embodiments, a Raman scanner scans a field of view of about 5×5 cm, 10×10 cm, 20×20 cm, or larger with a resolution of about 0.5 mm to about 5 mm. In some embodiments, a Raman scanner can include multiple excitation lasers, e.g., to improve acquisition speed. For example, the use of 4 lasers can reduce imaging time down to about 5 min. The Raman spectra can be delivered to a computer system described herein, e.g., via fiberoptics. In some embodiments, the computer system produces an image that can be overlayed on a photograph of the same field of view for anatomic coregistration.

In some embodiments, acquisition speed of a Raman scanner can be increased by increasing the number of the lasers, and/or acquiring using a line-laser technology (see, e.g., StreamLine™ Plus Raman imaging system, Renishaw Inc., Hoffman Estates, Ill.). In some embodiments, a scanner surface of the Raman scanner is configured to be brought into contact with a surface (e.g., a bed) to equalize the distances of the object to the focal point.

### Excitation Sources

Generally, excitation light for producing Raman photon scattering from a target cell and/or tissue is provided using a laser. Particular wavelengths useful in producing Raman scattering can be determined by the target to be excited. In some embodiments, excitation light is in the visible to near infrared range (e.g., about 400 nm to about 1400 nm). For example, in some embodiments, excitation light of 244 nm, 325 nm, 442 nm, 488 nm, 514 nm, 532 nm, 633 nm, 785 nm, or 830 nm can be used.

Selection of a particular wavelength for excitation light can be based on the particular substance to be excited. In some embodiments, a Raman nanoparticle, e.g., a SERS nanoparticle, is excited to produce Raman scattered photons. The composition of a particular Raman nanoparticle can be used to select an appropriate wavelength. In some embodiments, a SERS nanoparticle described in Kircher et al., Nature Med. 18:829-834 (2012); Yigit et al., Am. J. Nucl. Med. Mol. Imaging 2:232-241 (2012); Zhang et al., Small. 7:3261-9 (2011); or Zhang et al., Curr. Pharm. Biotechnol. 11:654-661 (2010) is used, and excitation light of 785 nm is used.

In some embodiment, an intrinsic non-enhanced or intrinsic enhanced (SERS) Raman spectrum of a tissue to be destroyed is excited. In such embodiments, selection of a particular wavelength of excitation light can be determined by particular properties of the diseased tissue.

### Detectors

Raman scattered photons from an illuminated sample can be collected and transmitted to one or more detectors. The detector(s) may be or may include a charge-coupled device (CCD) image sensor, for example, a time-gated intensified CCD camera (e.g., an ICCD camera). Alternatively or additional, the detector(s) may include an active pixel sensor (CMOS), an electron-multiplying CCD (EMCCD), frame transfer CCD, or the like.

In some embodiments, electromagnetic radiation used to obtain Raman images is transmitted to a detector in a “mappable” or “addressable” fashion, such that radiation (e.g., light) transmitted from different assessed regions of tissue can be differentiated by the detector. Light detected by a detector can be light transmitted, reflected, emitted, or scattered by the tissue through air interposed between the tissue surface and the detector. Alternatively, light can be transmitted by way of one or more optical fibers to the detector, for example. In some embodiments, one or more additional optical elements can be interposed between a target cell and/or tissue and detector(s). If optical elements are used to facilitate transmission from the surface to the detectors, other optical element(s) can be optically coupled with the fibers on either end or in the middle of such fibers. Examples of suitable optical elements include one or more lenses, beam splitters, diffraction gratings, polarization filters, bandpass filters, or other optical elements selected for transmitting or modifying light to be assessed by detectors. One or more appropriate optical elements may be coupled with a detector.

For example, a suitable filter can be a cut-off filter, a Fabry Perot angle tuned filter, an acousto-optic tunable filter, a liquid crystal tunable filter, a Lyot filter, an Evans split element liquid crystal tunable filter, a Solc liquid crystal tunable filter, or a liquid crystal Fabry Perot tunable filter. Suitable interferometers include a polarization-independent imaging interferometer, a Michelson interferometer, a Sagnac interferometer, a Twynam-Green interferometer, a Mach-Zehnder interferometer, and a tunable Fabry Perot interferometer.

### Tissue Ablation/Resection

As discussed herein, after a Raman signal is detected from cells and/or tissue, such cells and/or tissue are ablated or resected using known implements and/or methods for ablating or resecting cells and/or tissues, such as laser ablation, mechanical ablation, electro-cautery, radiofrequency ablation, and/or cryoablation.

In some embodiments, ablation is achieved using radiofrequency energy. Additional forms of energy for ablation include, without limitation, microwave energy, or photonic or radiant sources such as infrared or ultraviolet light. Photonic sources can include, for example, semiconductor emitters, lasers, and other such sources. Light energy may be either collimated or non-collimated. In some embodiments, ablation utilizes heatable fluids, or, alternatively, a cooling medium, including such non-limiting examples as liquid nitrogen, Freon™, non-CFC refrigerants, CO₂ or N₂O as an ablation energy medium. For ablations using hot or cold fluids or gases, an apparatus can be used to circulate heating/cool medium from outside a patient to a heating/cooling balloon or other element and then back outside the patient again. Mechanisms for circulating media in cryosurgical probes are well known in the ablation arts. For example, and incorporated by reference herein, suitable circulating mechanisms are disclosed in U.S. Pat. No. 6,182,666; U.S. Pat. No. 6,193,644; U.S. Pat. No. 6,237,355; and U.S. Pat. No. 6,572,610.

In some embodiments, light energy is used to ablate cells and/or tissues, and laser light is precisely aimed to cut or destroy diseased cells and/or tissue (e.g., a tumor) according to methods of the disclosure. In some embodiments, a method, system or device described herein is used to delivery laser-induced interstitial thermotherapy (LITT), or interstitial laser photocoagulation to target cells or tissues. LITT uses heat to shrink tumors by damaging or killing cancer cells. In some embodiments, a method, system or device described herein is used to delivery photodynamic therapy (PDT). In PDT, a certain drug (e.g., a photosensitizer or photosensitizing agent) is injected into a patient and absorbed by cells all over the patient's body. After a couple of days, the agent is found mostly in cancer cells. Laser light is then used to activate the agent and destroy cancer cells.

Lasers typically used to destroy cancerous tumors include solid state lasers, gas lasers, semiconductor lasers, and others. Typical wavelengths of electromagnetic radiation used in cancer treatments are from about 200 nm to about 5000 nm, and to about 12 μm for CO₂ lasers. Typical power levels range from about 0.1 W to about 15 W, and to about 30 W for CO₂ lasers. However, greater or lesser power levels may be used in some circumstances. Typical treatment times for exposing cancerous cells to laser energy range from less than about 1 minute to greater than about 1 hour, although longer or shorter times may be used. The laser energy applied to the cancerous cells may also be modulated. Laser energy may be applied to cancerous cells by continuous wave (constant level), pulsing (on/off), ramping (from low to high energy levels, or from high to low energy levels), or other waveforms (such as sine wave, square wave, triangular wave, etc.). Modulation of laser energy may be achieved by modulating energy to the laser light source or by blocking or reducing light output from the laser light source according to a desired modulation pattern.

Specific lasers for ablation of cells and/or tissues are known in the art. Exemplary, nonlimiting lasers useful in the methods, systems, and devices described herein include carbon dioxide (CO₂) lasers, argon lasers, and neodymium:yttrium-aluminum-garnet (Nd:YAG) lasers.

In some embodiments, cells and/or tissues are resected mechanically using, e.g., an electrically powered rotary blade. Additional mechanical resection mechanisms and/or methods may also be used. Resection mechanisms may include, for example, drills, dermatomes, scalpels, lancets, drill bits, rasps, trocars, and the like.

Other surgical instruments may be used in conjunction with the ablation and resection mechanisms described above, including, for example forceps, clamps, retractors, dilators, suction tips and tubes, irrigation needles, injection needles, calipers, and the like.

### Cells/Tissues

The methods, systems, and devices described herein can be used to resect and/or ablate a variety of cells and/or tissues, e.g., diseased cells and/or tissues. In some embodiments, methods described herein resect and/or ablate hyperproliferative, hyperplastic, metaplastic, dysplastic, and pre-neoplastic tissues.

By “hyperproliferative tissue” is meant a neoplastic cell growth or proliferation, whether malignant or benign, including all transformed cells and tissues and all cancerous cells and tissues. Hyperproliferative tissues include, but are not limited to, precancerous lesions, abnormal cell growths, benign tumors, malignant tumors, and cancer. Additional nonlimiting examples of hyperproliferative tissues include neoplasms, whether benign or malignant, located in the brain, prostate, colon, abdomen, bone, breast, digestive system, liver, pancreas, peritoneum, endocrine glands (adrenal, parathyroid, pituitary, testicles, ovary, thymus, thyroid), eye, head and neck, nervous (central and peripheral), lymphatic system, pelvic, skin, soft tissue, spleen, thoracic, or urogenital tract.

As used herein, the term “tumor” or “tumor tissue” refers to an abnormal mass of tissue that results from excessive cell division. A tumor or tumor tissue comprises “tumor cells”, which are neoplastic cells with abnormal growth properties and no useful bodily function. Tumors, tumor tissue, and tumor cells may be benign or malignant. A tumor or tumor tissue can also comprise “tumor-associated non-tumor cells”, such as vascular cells that form blood vessels to supply the tumor or tumor tissue. Non-tumor cells can be induced to replicate and develop by tumor cells, for example, induced to undergo angiogenesis within or surrounding a tumor or tumor tissue.

As used herein, the term “malignancy” refers to a non-benign tumor or a cancer. As used herein, the term “cancer” means a type of hyperproliferative disease that includes a malignancy characterized by deregulated or uncontrolled cell growth. Examples of cancer include, but are not limited to, carcinoma, lymphoma, blastoma, sarcoma, and leukemia or lymphoid malignancies. More particular examples of such cancers are noted below and include squamous cell cancer (e.g., epithelial squamous cell cancer), lung cancer (including small-cell lung cancer, non-small cell lung cancer, adenocarcinoma of the lung and squamous carcinoma of the lung), cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial cancer, uterine carcinoma, salivary gland carcinoma, kidney or renal cancer, prostate cancer, vulval cancer, thyroid cancer, hepatic carcinoma, anal carcinoma, penile carcinoma, as well as head and neck cancer. The term “cancer” includes primary malignant cells or tumors (e.g., those whose cells have not migrated to sites in the subject's body other than the site of the original malignancy or tumor) and secondary malignant cells or tumors (e.g., those arising from metastasis, the migration of malignant cells or tumor cells to secondary sites that are different from the site of the original tumor).

The methods described herein can also be used to ablate and/or resect premalignant tissue and to prevent progression to a neoplastic or malignant state including, but not limited to, those disorders described above. Such uses are indicated in conditions known or suspected of preceding progression to neoplasia or cancer, in particular where non-neoplastic cell growth consisting of hyperplasia, metaplasia, or dysplasia has occurred (see, e.g., Robbins and Angell, Basic Pathology, 2d Ed., W.B. Saunders Co., Philadelphia, pp. 68-79 (1976)).

The methods described herein can further be used to ablate and/or resect hyperplastic tissue. Hyperplasia is a form of controlled cell proliferation, involving an increase in cell number in a tissue or organ, without significant alteration in structure or function. Hyperplastic disorders include, but are not limited to, angiofollicular mediastinal lymph node hyperplasia, angiolymphoid hyperplasia with eosinophilia, atypical melanocytic hyperplasia, basal cell hyperplasia, benign giant lymph node hyperplasia, cementum hyperplasia, congenital adrenal hyperplasia, congenital sebaceous hyperplasia, cystic hyperplasia, cystic hyperplasia of the breast, denture hyperplasia, ductal hyperplasia, endometrial hyperplasia, fibromuscular hyperplasia, focal epithelial hyperplasia, gingival hyperplasia, inflammatory fibrous hyperplasia, inflammatory papillary hyperplasia, intravascular papillary endothelial hyperplasia, nodular hyperplasia of prostate, nodular regenerative hyperplasia, pseudoepitheliomatous hyperplasia, senile sebaceous hyperplasia, and verrucous hyperplasia.

The methods described herein can also be used to ablate and/or resect metaplastic tissue. Metaplasia is a form of controlled cell growth in which one type of adult or fully differentiated cell substitutes for another type of adult cell. Metaplastic disorders include, but are not limited to, agnogenic myeloid metaplasia, apocrine metaplasia, atypical metaplasia, autoparenchymatous metaplasia, connective tissue metaplasia, epithelial metaplasia, intestinal metaplasia, metaplastic anemia, metaplastic ossification, metaplastic polyps, myeloid metaplasia, primary myeloid metaplasia, secondary myeloid metaplasia, squamous metaplasia, squamous metaplasia of amnion, and symptomatic myeloid metaplasia.

The methods described herein can also be used to ablate and/or resect dysplastic tissue. Dysplasia can be a forerunner of cancer and is found mainly in the epithelia. Dysplasia is a disorderly form of non-neoplastic cell growth, involving a loss in individual cell uniformity and in the architectural orientation of cells. Dysplastic cells can have abnormally large, deeply stained nuclei, and exhibit pleomorphism. Dysplasia can occur, e.g., in areas of chronic irritation or inflammation. Dysplastic disorders include, but are not limited to, anhidrotic ectodermal dysplasia, anterofacial dysplasia, asphyxiating thoracic dysplasia, atriodigital dysplasia, bronchopulmonary dysplasia, cerebral dysplasia, cervical dysplasia, chondroectodermal dysplasia, cleidocranial dysplasia, congenital ectodermal dysplasia, craniodiaphysial dysplasia, craniocarpotarsal dysplasia, craniometaphysial dysplasia, dentin dysplasia, diaphysial dysplasia, ectodermal dysplasia, enamel dysplasia, encephalo-ophthalmic dysplasia, dysplasia epiphysialis hemimelia, dysplasia epiphysialis multiplex, dysplasia epiphysialis punctata, epithelial dysplasia, faciodigitogenital dysplasia, familial fibrous dysplasia of the jaws, familial white folded dysplasia, fibromuscular dysplasia, fibrous dysplasia of bone, florid osseous dysplasia, hereditary renal-retinal dysplasia, hidrotic ectodermal dysplasia, hypohidrotic ectodermal dysplasia, lymphopenic thymic dysplasia, mammary dysplasia, mandibulofacial dysplasia, metaphysial dysplasia, Mondini dysplasia, monostotic fibrous dysplasia, mucoepithelial dysplasia, multiple epiphysial dysplasia, oculoauriculovertebral dysplasia, oculodentodigital dysplasia, oculovertebral dysplasia, odontogenic dysplasia, ophthalmomandibulomelic dysplasia, periapical cemental dysplasia, polyostotic fibrous dysplasia, pseudoachondroplastic spondyloepiphysial dysplasia, retinal dysplasia, septo-optic dysplasia, spondyloepiphysial dysplasia, and ventriculoradial dysplasia.

Additional pre-neoplastic tissue that can be ablated and/or resected by the methods described herein include, but are not limited to, benign dysproliferative disorders (e.g., benign tumors, fibrocystic conditions, tissue hypertrophy, intestinal polyps, colon polyps, and esophageal dysplasia), leukoplakia, keratoses, Bowen's disease, Farmer's Skin, solar cheilitis, and solar keratosis.

The methods, systems, and devices described herein can also be used to resect and/or ablate infected cells and/or tissues. In some embodiments, methods described herein resect and/or ablate tissues infected with a virus, bacterium, fungus, protozoan, and/or helminth.

In some embodiments, infected tissue is infected with one or more of an immunodeficiency virus (e.g., a human immunodeficiency virus (HIV), e.g., HIV-1, HIV-2), a hepatitis virus (e.g., hepatitis B virus (HBV), hepatitis C virus (HCV), hepatitis A virus, non-A and non-B hepatitis virus), a herpes virus (e.g., herpes simplex virus type I (HSV-1), HSV-2, Varicella-zoster virus, Epstein Barr virus, human cytomegalovirus, human herpesvirus 6 (HHV-6), HHV-7, HHV-8), a poxvirus (e.g., variola, vaccinia, monkeypox, Molluscum contagiosum virus), an influenza virus, a human papilloma virus, adenovirus, rhinovirus, coronavirus, respiratory syncytial virus, rabies virus, coxsackie virus, human T-cell leukemia virus (types I, II and III), parainfluenza virus, paramyxovirus, poliovirus, rotavirus, rhinovirus, rubella virus, measles virus, mumps virus, adenovirus, yellow fever virus, Norwalk virus, West Nile virus, a Dengue virus, Severe Acute Respiratory Syndrome Coronavirus (SARS-CoV), bunyavirus, Ebola virus, Marburg virus, Eastern equine encephalitis virus, Venezuelan equine encephalitis virus, Japanese encephalitis virus, St. Louis encephalitis virus, Junin virus, Lassa virus, and Lymphocytic choriomeningitis virus.

In some embodiments, infected tissue is infected with one or more bacteria from the following genera and species: Chlamydia(e.g., Chlamydia pneumoniae, Chlamydia psittaci, Chlamydia trachomatis), Legionella(e.g., Legionella pneumophila), Listeria(e.g., Listeria monocytogenes), Rickettsia(e.g., R. australis, R. rickettsii, R. akari, R. conorii, R. sibirica, R. japonica, R. africae, R. typhi, R. prowazekii), Actinobacter (e.g., Actinobacter baumannii), Bordetella(e.g., Bordetella pertussis), Bacillus(e.g., Bacillus anthracis, Bacillus cereus), Bacteroides(e.g., Bacteroides fragilis), Bartonella(e.g., Bartonella henselae), Borrelia(e.g., Borrelia burgdorferi), Brucella(e.g., Brucella abortus, Brucella canis, Brucella melitensis, Brucella suis), Campylobacter(e.g., Campylobacter jejuni), Clostridium(e.g., Clostridium botulinum, Clostridium difficile, Clostridium perfringens, Clostridium tetani), Corynebacterium(e.g., Corynebacterium diphtheriae, Corynebacterium amycolatum), Enterococcus(e.g., Enterococcus faecalis, Enterococcus faecium), Escherichia(e.g., Escherichia coli), Francisella(e.g., Francisella tularensis), Haemophilus(e.g., Haemophilus influenzae), Helicobacter(e.g., Helicobacter pylori), Klebsiella(e.g., Klebsiella pneumoniae), Leptospira (e.g., Leptospira interrogans), Mycobacteria (e.g., Mycobacterium leprae, Mycobacterium tuberculosis), Mycoplasma(e.g., Mycoplasma pneumoniae), Neisseria(e.g., Neisseria gonorrhoeae, Neisseria meningitidis), Pseudomonas(e.g., Pseudomonas aeruginosa), Salmonella(e.g., Salmonella typhi, Salmonella typhimurium, Salmonella enterica), Shigella(e.g., Shigella dysenteriae, Shigella sonnei), Staphylococcus(e.g., Staphylococcus aureus, Staphylococcus epidermidis, Staphylococcus saprophyticus), Streptococcus(e.g., Streptococcus agalactiae, Streptococcus pneumoniae, Streptococcus pyogenes), Treponoma (e.g., Treponoma pallidum), Vibrio(e.g., Vibrio cholerae, Vibrio vulnificus), and Yersinia(e.g., Yersinia pestis).

In some embodiments, infected tissue is infected with one or more protozoa, for example, one or more of Cryptosporidium parvum, Entamoeba(e.g., Entamoeba histolytica), Giardia(e.g., Giardia lambila), Leishmania(e.g., Leishmania donovani), Plasmodium spp. (e.g., Plasmodium falciparum, Plasmodium vivax, Plasmodium ovale, Plasmodium malariae), Toxoplasma(e.g., Toxoplasma gondii), Trichomonas(e.g., Trichomonas vaginalis), and Trypanosoma(e.g., Trypanosoma brucei, Trypanosoma cruzi).

In some embodiments, infected tissue is infected with one or more fungal pathogens such as Aspergillus, Candida(e.g., Candida albicans), Coccidiodes (e.g., Coccidiodes immitis), Cryptococcus(e.g., Cryptococcus neoformans), Histoplasma(e.g., Histoplasma capsulatum), and Pneumocystis(e.g., Pneumocystis carinii).

In some embodiments, infected tissue is infected with one or more helminths, such as Ascaris lumbricoides, Ancylostoma, Clonorchis sinensis, Dracuncula medinensis, Enterobius vermicularis, Filaria, Onchocerca volvulus, Loa loa, Schistosoma, Strongyloides, Trichuris trichura, and Trichinella spiralis.

### Computer/Software

Embodiments may include a computer which executes software that controls the operation of one or more instruments/devices, and/or that processes data obtained by the system. The software may include one or more modules recorded on machine-readable media such as magnetic disks, magnetic tape, CD-ROM, and semiconductor memory, for example. The machine-readable medium may be resident within the computer or can be connected to the computer by a communication link (e.g., access via internet link). However, in alternative embodiments, one can substitute computer instructions in the form of hardwired logic for software, or one can substitute firmware (i.e., computer instructions recorded on devices such as PROMs, EPROMS, EEPROMs, or the like) for software. The term machine-readable instructions as used herein is intended to encompass software, hardwired logic, firmware, object code and the like.

The computer can be, for example, a general purpose computer. The computer can be, for example, an embedded computer, a personal computer such as a laptop or desktop computer, or another type of computer, that is capable of running the software, issuing suitable control commands, and/or recording information in real-time. The computer may include a display for reporting information to an operator of the system/device (e.g., displaying a view field to a surgeon during an operation), a keyboard and/or other I/O device such as a mouse for enabling the operator to enter information and commands, and/or a printer for providing a print-out. In certain embodiments, some commands entered at the keyboard enable a user to perform certain data processing tasks.

### Auxiliary Imaging Systems

The Raman-based systems, methods, and devices described herein that are utilized in a surgical or non-surgical procedure may be used in combination with other imaging systems implemented before, during, or after the procedure. For example, the Raman-based systems, methods, and devices may be used in combination with video, microscope, x-ray, Computed Tomography (CT), magnetic resonance imaging (MRI), ultrasound (US), thermography, fluorescence imaging, Diffuse Optical Tomography (DOT), Positron Emission Tomography (PET), PET/CT, Single Photon Emission Computed Tomography (SPECT), and/or SPECT/CT systems.

In some embodiments, a target tissue (e.g., diseased tissue) is imaged using an auxiliary imaging system, and the image can be used to guide a Raman ablation system described herein to the target tissue. In some embodiments, an auxiliary imaging system includes hardware and/or software for co-registering the image with detected Raman signals. For example, a video camera can be used in conjunction with the Raman system described herein, such that the video camera provides an image that serves to identify locations at which the ablation or resection device is inoperative (regardless of the presence of a Raman reporter at such location). Furthermore, other detection modalities, such as MRI, NMR, PET, SPECT, CT, X-ray, ultrasound, photoacoustic detection, and/or fluorescent detection can be used in conjunction with the Raman systems described herein to identify tissue to be resected/ablated.

### EQUIVALENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A system comprising:
an excitation light source for directing excitation light onto or into a target tissue;
an instrument operably linked to the excitation light source, the instrument comprising:
optics for directing the excitation light onto or into the target tissue;
a detector for detecting Raman scattered photons emanating from the target tissue, said Raman scattered photons resulting from illumination with the excitation light and
a resector/ablator mechanism;
a processor configured to process data corresponding to the Raman scattered photons detected from the target tissue; and
a resector/ablator controller operably linked to the processor and operably linked to the resector/ablator mechanism.

2. The system of claim 1, wherein the excitation light source is a laser.

3. The system of claim 1, wherein the excitation light has a wavelength of about 500 nm to about 10 μm.

4. The system of claim 1, wherein the instrument is an endoscopic instrument.

5. The system of claim 1, wherein the instrument comprises optics for imaging.

6. The system of claim 1, wherein the resector/ablator mechanism comprises a laser.

7. The system of claim 6, wherein the laser of the resector/ablator mechanism is a CO2 laser.

8. The system of claim 1, wherein the resector/ablator mechanism is a mechanical resector, an electro-cautery mechanism, a cryoablation mechanism, and/or a radiofrequency ablation mechanism.

9. The system of claim 1, wherein the resector/ablator controller is configured to activate the resector/ablator mechanism to resect, ablate, and/or destroy tissue at a given location only if Raman scattered photons detected from the given location indicate the presence of a Raman reporter.

10. The system of claim 1, further comprising a suction vacuum operably linked to the instrument.

11. A method of resecting, ablating, and/or destroying diseased tissue, the method comprising:
positioning an instrument in relation to a first location of a target tissue of a subject, the instrument comprising:
optics for directing excitation light onto or into the target tissue at a given location;
a detector for detecting Raman scattered photons emanating from the target tissue at the given location; and
a resector/ablator mechanism;
detecting the Raman scattered photons emanating from the first location of the target tissue;
analyzing the detected Raman scattered photons emanating from the first location to determine whether the detected photons are indicative of the presence of a Raman reporter at the first location; and
activating the resector/ablator mechanism to resect the target tissue at the first location only if the analyzed photons from the first location are determined to be indicative of the presence of the Raman reporter at the first location.

12. The method of claim 11, further comprising:
deactivating the resector/ablator mechanism prior to repositioning of the instrument in relation to a second location of the target tissue;
detecting the Raman scattered photons emanating from the second location of the target tissue;
analyzing the detected Raman scattered photons emanating from the second location to determine whether the detected photons are indicative of the presence of a Raman reporter at the second location; and
activating the resector/ablator mechanism to resect, ablate, and/or destroy the target tissue at the second location only if the analyzed photons from the second location are determined to be indicative of the presence of the Raman reporter at the second location.

13. The method of claim 11, further comprising administering nanoparticles to the subject prior to implementation of the instrument.

14. The method of claim 11, further comprising scanning the subject prior to implementation of the instrument to confirm the absence of nanoparticles from healthy (e.g., normal, non-cancerous) tissue.

15. The method of claim 11, wherein the instrument is operably linked to an excitation light source.

16. The method of claim 15, wherein the excitation light source is a laser.

17. The method of claim 11, wherein the excitation light has a wavelength of about 500 nm to about 10 μm.

18. The method of claim 11, wherein the instrument is an endoscopic device.

19. The method of claim 11, wherein the instrument comprises optics for imaging.

20. The method of claim 11, wherein the resector/ablator mechanism comprises a laser.

21. The method of claim 20, wherein the laser of the resector/ablator mechanism is a CO2 laser.

22. The method of claim 11, wherein the resector/ablator mechanism is a mechanical resector, an electro-cautery mechanism, a cryoablation mechanism, and/or a radiofrequency ablation mechanism.

23. The method of claim 11, wherein the analyzing step comprises using a computer processor (e.g., a Raman spectrometer and associated computer processor and/or software) to process data corresponding to the detected Raman scattered photons.

24. The method of claim 11, further comprising removing resected tissue.

25. The method of claim 11, wherein the method is an in vivo method.

26. A method of resecting, ablating, and/or destroying diseased tissue, the method comprising:
positioning a resector/ablator instrument in relation to a first location of a target tissue of a subject;
detecting Raman scattered photons emanating from the first location of the target tissue using the instrument;
analyzing the detected Raman scattered photons emanating from the first location to determine whether the detected photons are indicative of the presence of a Raman reporter at the first location; and
resecting and/or ablating, via the instrument, the target tissue at the first location only if the analyzed photons from the first location are determined to be indicative of the presence of the Raman reporter at the first location.
